(19) United States
(12) Patent Application Publication
(10) Pub. No.: US 2007/0166262 A1
(43) Pub. Date: Jul. 19, 2007

(54) Leave-on hair care composition

(76) Inventors: Stuart Keith Pratley, Wirral (GB); Aart Van Vuure, Randwijk (NL)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/571,641

(22) PCT Filed: Aug. 27, 2004
(86) PCT No.: PCT/EP04/09611

§ 371 Date: Nov. 13, 2006

(30) Foreign Application Priority Data
Sep. 15, 2003 (EP) 03255746.4
Nov. 28, 2003 (EP) 03257513.6

(51) Int. Cl. A61K 8/37; A61K 8/81

(52) U.S. Cl. 424/70.12; 424/70.31

(57) ABSTRACT

A leave on hair care composition comprising an oil in water emulsion wherein the oil phase comprises: (i) one or more non-volatile oils, where the dynamic viscosity of any single non-volatile oil or the dynamic viscosity of a blend of non-volatile oils is less than 1000 mPa.s at 25° C. and 5 s⁻¹; (ii) an oil dispersible structurant such that the dynamic viscosity of the structured oil phase at 25° C. prior to formation of the emulsion is at least 50,000 mPa.s at a shear rate of 0.5 s⁻¹ and is less than 6,000 mPa.s at a shear rate of 500 s⁻¹.

### FIELD OF THE INVENTION

This invention relates to hair care compositions, in particular to hair care compositions that style the hair.

### BACKGROUND AND PRIOR ART

Hair styling products are widely used and are usually applied in the form of, sprays, mousses, gels and lotions. A major disadvantage with using styling creams is that they have a tendency to feel sticky both in the pot before styling, on the hands when applying and on the hair after styling.

The present invention is a hair styling cream that helps mitigate the problem of stickiness.

Further advantages of the creams of the present invention are: they can be used to maintain a style; enable re-styling of hair; condition hair; reduce hair damage; provide hair shine; provide a wet look; control hair volume and align the hair.

### DESCRIPTION OF THE PRESENT INVENTION

According to the present invention, there is provided a leave on hair care composition comprising an oil in water emulsion wherein the oil phase comprises:
i) one or more non-volatile oils, where the dynamic viscosity of any single non-volatile oil or the dynamic viscosity of a blend of non-volatile oils is less than 1000 mPa.s at 25° C. and 5 s⁻¹.
ii) an oil dispersible structurant such that the dynamic viscosity of the oil phase at 25° C. prior to formation of the emulsion is at least 50,000 mPa.s at a shear rate of 0.5 s⁻¹, and is less than 6,000 mPa.s at a shear rate of 500 s⁻¹.

The invention also relates to a method of styling hair by applying to the hair a composition as described above.

Further described is a method of treating hair with a hair care composition comprising an oil-in-water emulsion containing a structured oil phase such that after application part of the composition evaporates or absorbs into the hair to leave a residue on the hair,

where the residue has a dynamic viscosity at 25° C. of at least 50,000 mPa.s at a shear rate of 0.5 s⁻¹, and less than 6,000 mpa.s at a shear rate of 500 s−1.

The invention also relates to the use of a hair composition as described above for styling hair, maintaining a style, enabling re-styling of hair, conditioning hair, reducing hair damage, providing hair shine, providing a wet look and/or controlling hair volume or alignment.

### DETAILED DESCRIPTION OF THE INVENTION

Although the composition may be any leave on formulation it is preferable if it is in the form of a cream. In the context of the present invention a cream is defined as not immediately pourable under gravity.

It is preferable if the dynamic viscosity of the final composition is from 2,000 to 300,000 mpa.s at 5 s−1 and 25° C., more preferably from 10,000 to 300,000 mPa.s, most preferably from 20,000 to 200,000 mPa.s. It is highly preferable if the dynamic viscosity is from 30,000 to 150,000 mpa.s

The dynamic viscosity of the fluids was determined with a standard stress controlled rheometer (this case Carrimed CSL-100), using a parallel plate configuration at a gap height of 200 μm and by measuring dynamic viscosity at a range of shear rates.

The Oil Phase

It is advantageous, for styling hair, if the oil phase at 25° C. prior to addition to the cream has a yield stress of at least 500 Pa, preferably a yield stress of at least 1,000 Pa.

The yield stress of a fluid is defined as the critical stress at which a sharp drop in dynamic viscosity is observed, typically a few orders of magnitude of dynamic viscosity drop when the stress is increasing less than an order of 2. The yield stress is associated with a collapse in the structure of the fluid.

It is also preferable if the oil phase prior to formation of the emulsion when sheared to a point past its yield point has a recovery ratio of at least 5%, more preferably at least 10% and most preferably at least 15%.

The recovery ratio of the structured oil phase is defined in the context of the present invention as the dynamic viscosity of the oil phase at a shear rate of 0.5 s⁻¹ after having been sheared beyond its yield stress (the maximum shear rate used to induce yield was 500 s⁻¹), normalised to the dynamic viscosity of the oil phase at a shear rate of 0.5 s⁻¹ before having been sheared beyond its yield point. Thus the measuring protocol was to measure the dynamic viscosity whilst ramping up the shear rate from 0.5 s⁻¹ to 500 s⁻¹ over a period of 5 minutes, immediately followed by a shear rate ramp down from 500 s⁻¹ to 0.5 s⁻¹ over 5 minutes.

It is beneficial if the residue of the composition after application on hair, when part of the composition evaporates or absorbs into the hair, has Theological properties, (dynamic viscosities at 0.5 and 500 s−1, yield stress and recovery ratio) at 25° C. which are within a factor of 2 (either an increase or decrease) of the Theological properties of the oil phase prior to addition to the cream.

The residue of the cream is defined as the material, which stays behind on the hair after application of the cream to hair. After application of the cream the water in the cream will typically be absorbed by the hair and any volatiles in the cream will evaporate. It is understood that the water in the cream is absorbed by the hair within a few minutes after application, if the cream is applied to dry or slightly damp hair. To simulate the residue, cream is being dried in a petri-dish at ambient temperature in standard conditions (20° C. and 50% RH) for a few days. The cream residue typically contains less than 10% of remaining water.

Non-Volatile Oils

The composition comprises one or more non-volatile oils, where the dynamic viscosity of any single non-volatile oil or the dynamic viscosity of a blend of non-volatile oils is less than 1000 mPa.s at 25° C. at 5 s⁻¹. Preferably the dynamic viscosity is less than 500 mpa.s at 25° C. at 5 s⁻¹, more preferably less than 100 mPa.s, most preferably less than 50 mPa.s.

A non-volatile oil is defined such that when the emollient is placed in a petri-dish in a room at standard environmental conditions (20C, 50% RH) at a fluid height of 3 mm, after 1 hour less than 10 wt % of the emollient will have evaporated.

Oils can be selected from the group consisting of triglycerides, fatty esters, fatty alcohols, fatty acids or mineral oils (branched hydrocarbons) and mixtures thereof.

Preferably the non-volatile oil is selected from the group consisting of triheptanoin, tricaprylin, tricaprin, triundecanoin, trilinolein, triolein, almond oil, coconut oil, olive oil, palm kernel oil, peanut oil, sunflower oil, isopropylmyristate, isopropylpalmitate, isocetyl stearate, ethyl oleate, octyl isostearate, butyl myristate, butyl stearate, octyl palmitate, ethylhexyl cocoate, octyl dodecanol, oleyl alcohol, isostearyl alcohol, isostearic acid, mineral oil, paraffin oil, dicaprylate/dicaprate propylene glycol, C12-15 alkyl benzoate and derivatives or mixtures thereof.

Suitable mineral oils are those sold under the name Sirius White Oils by Fuchs Lubricants (UK). Examples of suitable oils are Sirius M85, Sirius M125 and Sirius M350.

Other suitable oils are silicone oils, for example dimethicone, with dynamic viscosity as previously defined.

The level of the oil phase within the total composition is preferably greater than 10 wt % of the total composition, more preferably greater than 20 wt %.

Oil Structurant

The structurant is oil dispersible.

Suitable structurants are selected from the group consisting of dextrin palmitate, trihydroxystearin, hydroxy stearic acid, hydrophilic or hydrophobic silica or preferably a hydrophobically modified clay such as stearalkonium hectorite, quaternium-18 bentonite, quaternium-18 hectorite or disteardimonium hectorite and derivatives or mixtures of these.

If the structurant is a hydrophobically modified clay, such as stearalkonium hectorite, it is preferable if the oil phase further contains a polar activator. A polar activator polarises the edges of the hydrophobically modified clay platelets so the clay platelets form a network structure when dispersed in oil through polar interaction. Suitable polar activators are propylene carbonate preferably in water or an ethanol/water 95:5 mixture. The level of activator is preferably from 1 to 10 wt. %. The weight ratio of clay to activator is from 1.3 to 4.1 by weight more preferably from 2:1 to 4:1.

The structurant should be chosen such that the dynamic viscosity of the structured oil phase at 25° C. prior to addition to the emulsion is at least 50,000, more preferably at least 100,000, most preferably at least 250,000 mpa.s at a shear rate of 0.5 s⁻¹, and is less than 6,000, more preferably less than 4,000, most preferably less than 2,000 mpa.s at a shear rate of 500 s⁻¹ . A highly preferred oil phase has a dynamic viscosity at 25° C. of at least 500,000 mpa.s at a shear rate of 0.5 s⁻¹ and a dynamic viscosity of 1,000 mpa.s or less at a shear rate of 500 s⁻¹.

The level of structurant within the oil phase is preferably from 0.5 to 20 wt % more preferably from 1 to 15 wt. % most preferably from 2 to 10 wt. % of the total oil content in the formulation.

The level of structurant within the total formulation is preferably from 0.3 to 10 wt. %, more preferably 1 to 5 wt. %.

Surfactant System

The compositions of the invention are Oil-in-Water emulsions and usually require a suitable surfactant system to emulsify the structured oil material.

Particularly suitable for this purpose are non-ionic surfactants. To obtain an Oil-in-Water emulsion it is desirable to use a surfactant system with a high HLB (Hydrophilic to Lipophilic Balance) value. It is particularly desirable in this case to use a mixture of a high HLB surfactant and a low HLB surfactant where the composite surfactant system has high HLB. This way it is known to those skilled in the art that a strong surfactant film is formed at the Oil/Water interface.

There are many suitable non-ionic surfactant systems, but particularly preferred is a surfactant mixture of 80% polyoxyethylene sorbitan monostearate (20 EO) and 20% sorbitan monostearate.

Styling Compound

In some aspects of this invention it is desirable if the composition comprises an additional styling aid.

Particularly useful as styling aids with this invention are hair styling polymers. Hair styling polymers are well known articles of commerce and many such polymers are available commercially which contain moieties, which render the polymers cationic, anionic, amphoteric or nonionic in nature. The polymers may be synthetic or naturally derived.

The amount of the hair styling polymer may range from 0.1 to 10%, preferably 0.5 to 8%, more preferably 0.75 to 6% by weight based on total weight of the composition.

Examples of nonionic hair styling polymers are homopolymers of N-vinylpyrrolidone and copolymers of N-vinylpyrrolidone with compatible nonionic monomers such as vinyl acetate. Nonionic polymers containing N-vinylpyrrolidone in various weight average molecular weights are available commercially from ISP Corporation—specific examples of such materials are homopolymers of N-vinylpyrrolidone having an average molecular weight of about 630,000 sold under the name PVP K-90 and homopolymers of N-vinylpyrrolidone having an average molecular weight of about 1,000,000 sold under the name of PVP K-120. Particularly preferred is a copolymer of polyvinyl pyrrolidone and polyvinyl acetate. An example of this copolymer is sold by BASF under the name Luviskol VA64.

Examples of cationic hair styling polymers are copolymers of amino-functional acrylate monomers such as lower alkyl aminoalkyl acrylate, or methacrylate monomers such as dimethylaminoethyl methacrylate, with compatible monomers such as N-vinylpyrrolidone, vinyl caprolactam, alkyl methacrylates (such as methyl methacrylate and ethyl methacrylate) and alkyl acrylates (such as ethyl acrylate and n-butyl acrylate).

Specific examples of suitable cationic hair styling polymers are:
copolymers of N-vinylpyrrolidone and dimethylaminoethyl methacrylate, available from ISP Corporation as Copolymer 845, Copolymer 937 and Copolymer 958;
copolymers of N-vinylpyrrolidone and dimethylaminopropylacrylamide or methacrylamide, available from ISP Corporation as Styleze® CC10;
copolymers of N-vinylpyrrolidine and dimethylaminoethyl methacrylate;
copolymers of vinylcaprolactam, N-vinylpyrrolidone and dimethylaminoethylmethacrylate;
Polyquaternium-4 (a copolymer of diallyldimonium chloride and hydroxyethylcellulose);
Polyquaternium-11 (formed by the reaction of diethyl sulphate and a copolymer of vinyl pyrrolidone and dimethyl aminoethylmethacrylate), available from ISP as Gafquat® 734, 755 and 755N, and from BASF as Luviquat® PQ11;
Polyquaternium-16 (formed from methylvinylimidazolium chloride and vinylpyrrolidone), available from BASF as Luviquat® FC 370, FC 550, FC 905 and HM-552;
Polyquaternium-46 (prepared by the reaction of vinylcaprolactam and vinylpyrrolidone with methylvinylimidazolium methosulphate), available from BASF as Luviquat®Hold.

Examples of anionic hair styling polymers are:
copolymers of vinyl acetate and crotonic acid;
terpolymers of vinyl acetate, crotonic acid and a vinyl ester of an alpha-branched saturated aliphatic monocarboxylic acid such as vinyl neodecanoate;
copolymers of methyl vinyl ether and maleic anhydride (molar ratio about 1:1) wherein such copolymers are 50% esterified with a saturated alcohol containing from 1 to 4 carbon atoms such as ethanol or butanol;
acrylic copolymers containing acrylic acid or methacrylic acid as the anionic radical-containing moiety with other monomers such as: esters of acrylic or methacrylic acid with one or more saturated alcohols having from 1 to 22 carbon atoms (such as methyl methacrylate, ethyl acrylate, ethyl methacrylate, n-butyl acrylate, t-butyl acrylate, t-butyl methacrylate, n-butyl methacrylate, n-hexyl acrylate, n-octyl acrylate, lauryl methacrylate and behenyl acrylate); glycols having from 1 to 6 carbon atoms (such as hydroxypropyl methacrylate and hydroxyethyl acrylate); styrene; vinyl caprolactam; vinyl acetate; acrylamide; alkyl acrylamides and methacrylamides having 1 to 8 carbon atoms in the alkyl group (such as methacrylamide, t-butyl acrylamide and n-octyl acrylamide); and other compatible unsaturated monomers.

The additional styling polymer may also contain grafted silicone, such as polydimethylsiloxane.

Specific examples of suitable anionic hair styling polymers are:
RESYN® 28-2930 available from National Starch (vinyl acetate/crotonic acid/vinyl neodecanoate copolymer);
ULTRAHOLD® 8 available from BASF (CTFA designation Acrylates/acrylamide copolymer);
the GANTREZ®ES series available from ISP Corporation esterified copolymers of methyl vinyl ether and maleic anhydride).

Other suitable anionic hair styling polymers include carboxylated polyurethanes. Carboxylated polyurethane resins are linear, hydroxyl-terminated copolymers having pendant carboxyl groups. They may be ethoxylated and/or propoxylated at least at one terminal end. The carboxyl group can be a carboxylic acid group or an ester group, wherein the alkyl moiety of the ester group contains one to three carbon atoms. The carboxylated polyurethane resin can also be a copolymer of polyvinylpyrrolidone and a polyurethane, having a CTFA designation PVP/polycarbamyl polyglycol ester. Suitable carboxylated polyurethane resins are disclosed in EP-A-0619111 and U.S. Pat. No. 5,000,955.

Other suitable hydrophilic polyurethanes are disclosed in U.S. Pat. Nos. 3,822,238; 4,156,066; 4,156,067; 4,255,550; and 4,743,673.

Amphoteric hair styling polymers which can contain cationic groups derived from monomers such as t-butyl aminoethyl methacrylate as well as carboxyl groups derived from monomers such as acrylic acid or methacrylic acid can also be used in the present invention. One specific example of an amphoteric hair styling polymer is Amphomer® (Octylacrylamide/acrylates/butylaminoethyl methacrylate copolymer) sold by the National Starch and Chemical Corporation.

Examples of suitable naturally-derived hair styling polymers include shellac, alginates, gelatins, pectins, cellulose derivatives and chitosan or salts and derivatives thereof. Commercially available examples include Kytamer® (ex Amerchol) and Amaze® (ex National Starch).

Further Components

Styling products frequently include a carrier and further additional components. The carriers and additional components required to formulate such products vary with product type and can be routinely chosen by one skilled in the art. The following is a description of some of these carriers and additional components.

The composition preferably comprises an aqueous phase. An aqueous phase thickener is preferably present and can be based on a cellulose derivative, in particular hydroxyethyl cellulose or cetyl hydroxyethyl cellulose. An alternative aqueous phase thickener is carbomer. Such aqueous phase thickeners are typically present in an amount from 0.01% to 10% by weight.

Hair care compositions of the present invention can comprise a carrier, or a mixture of such carriers, which are suitable for application to the hair. The carriers are present at from about 0.5% to about 99.5%, preferably from about 5.0% to about 99.5%, more preferably from about 10.0% to about 98.0%, of the composition. As used herein, the phrase “suitable for application to hair” means that the carrier does not damage or negatively affect the aesthetics of hair or cause irritation to the underlying skin.

Compositions according to the invention comprise a buffer or pH adjuster. Preferred buffers or pH adjusters include weak acids and bases such as glycine/sodium hydroxide, citric acid, triethanolamine, lactic acid, succinic acid, acetic acid and salts thereof. Frequently a mixture of buffering system is used such as sodium citrate and citric acid.

Carriers suitable for use with hair care compositions of the present invention include, for example, those commonly used in creams. The carriers used herein can include a wide range of components conventionally used in hair care compositions. The carriers can contain a solvent to dissolve or disperse the styling compound being used, with water, the C₁-C₆ alcohols, lower alkyl acetate and mixtures thereof being preferred. The carriers can also contain a wide variety of additional materials such as acetone, hydrocarbons (such as isobutane, hexane, decene), water, ethanol, volatile silicone derivatives, and mixtures thereof. The solvents used in such mixtures may be miscible or immiscible with each other.

The carrier can include a wide variety of further conditioning materials suitable for hair such as quaternary silicone polymers, silicone based conditioners and their emulsions, and amino functional silicones and their emulsions. The dynamic viscosity of these conditioning silicones is greater than 10,000 mpa.s at 25° C. and 5 s⁻¹.

Further general ingredients suitable for all product forms include, sun-screening agents, preservatives, anti-oxidants, anti-dandruff actives, and emulsifiers for emulsifying the various carrier components of the compositions of the invention.

The compositions of the present invention may also contain adjuncts suitable for hair care. Generally such ingredients are included individually at a level of up to 2, preferably up to 1 wt % of the total composition. Suitable hair care adjuncts include amino acids, sugars and ceramides.

Compositions of the present invention are formulated into hair care compositions, especially products with hair styling claims. The compositions are for use in styling human hair and, more preferably, they are packaged and labeled as such.

It is preferred if the products are left on hair after application and not immediately washed off.

The following non-limiting Examples further illustrate the preferred embodiments of the invention. All percentages referred to in the examples and throughout this specification are by weight based on total weight unless otherwise indicated.

Examples of the invention are illustrated by a number, comparative examples are illustrated by a letter.

### EXAMPLES

Examples 1 and 2 comprise a structured oil phase, Example A does not

| | | wt % active ingredient | | |
|---|---|---|---|---|
| Trade Name | Chemical name | Example 1 | Example 2 | Example A |
| Trivent OCG | Tricaprylin | | 26.2 | 30 |
| Estol 1514 | Isopropylmyristate | 26.5 | | |
| Bentone 27V | Stearalkonium hectorite | 2.7 | 2.9 | |
| Propylene carbonate | 1,3-dioxolan-2-one, 4-methyl- | 0.8 | 0.9 | |
| Tween 60 | Polyoxyethylene sorbitan monostearate (20 EO) | 7.6 | 7.6 | 7.6 |
| Span 60 | Sorbitan monostearate | 1.9 | 1.9 | 1.9 |
| Carbopol 940 | Carbomer | 0.3 | 0.35 | 0.3 |
| Luviskol VA64W | PVP/PVA copolymer | | 3 | |
| Nipagin M | Methyl paraben | 0.2 | 0.2 | 0.2 |
| Nipasol M | Propyl paraben | 0.1 | 0.1 | 0.1 |
| BHT | Dibutylhydroxytoluene | 0.05 | 0.05 | 0.05 |
| Sepicide LD | Phenoxyethanol | 0.4 | 0.4 | 0.4 |
| Perfume | Perfume blend | 0.1 | 0.1 | 0.1 |
| Water | Distilled water | to 100 | to 100 | to 100 |

The above creams are all Oil-in-Water emulsions and they were prepared as follows:

The dynamic viscosity out of the tricaprylin (trivent OCG) is 20 mPas (25° C., 5 s⁻¹) and of isopropylmyristate (Estol 1514) 5mPas (25° C., 5s⁻¹)

For Examples 1 and 2 a 95/5. wt % mixture of propylene carbonate and water was prepared. Then the oil was heated to 50° C. and Bentone powder was added under high shear, after which the activator mixture was slowly added whilst mixing under high shear. (The dynamic viscosity of the bentone/oil mix was as follows: at 25° C. 0.5 s⁻¹ 340,000 mPas for Example 1 and 4,000,000 mPas for Example 2 at 25° C. and at 500 s⁻¹, 25° C. 330 mpas for Example 1 and 2,400 mPas for Example 2.) Carbomer and methyl paraben were mixed into the water phase under rapid agitation. Bentone gel and all oil phase ingredients (surfactants+preservatives) were mixed under low shear at 70° C. The water phase was preheated to 70° C. and then slowly emulsified into the oil phase under low shear and vacuum. In case PVP/PVA was used this was now added to the emulsion under low shear. High shear was employed for 7.5 minutes under vacuum. The emulsion was then cooled to 40° C. at 10° C./15 minutes under low shear. The pH was neutralised with a 5% solution of NaOH. Perfume was then added at 40° C. Finally high (post-)shear was conducted for 7.5 minutes under vacuum.

The Examples were applied to mannequin heads with short (average 8 cm) Caucasian hair. In the case of Example A it was impossible to create a very incongruent style with the hair standing up as the hair fibres fell back down almost immediately. However, for Examples 1 and 2 it was clearly possible to create such a style with the hair standing up. It was easier to achieve this styling effect when the styling polymer is also present, so Example 2 is easier to style with than Example 1.

Example 2 and Example A were evaluated by a qualitative consumer panel (12 people) and compared against a commercial benchmark product which has good styling performance. Example 2 was rated similar in styling performance to the benchmark product but the stickiness of the product on hair was rated much lower. Example A was rated as insufficient for styling whereas the sensory properties were rated favourably (comparable to Example 2). This illustrates the combination of good styling and good sensory performance for Example 2.

The following are also examples of stable formulations according to the invention. These Examples were used to style hair with acceptable stickiness.

| | | wt % active ingredient | |
|---|---|---|---|
| Trade Name | Chemical name | Example 3 | Example 4 |
| Trivent OCG | Tricaprylin | 36.7 | 34.8 |
| Hydroxy Stearic Acid | 12-Hydroxystearic acid | 3.3 | |
| Rheopearl KL | Dextrin palmitate | | 5.2 |
| Tween 60 | Polyoxyethylene sorbitan | 7.6 | 7.6 |
| | monostearate (20 EO) | | |
| Span 60 | Sorbitan monostearate | 1.9 | 1.9 |
| Carbopol 940 | Carbomer | 0.3 | 0.3 |
| Nipagin M | Methyl paraben | 0.2 | 0.2 |
| Nipasol M | Propyl paraben | 0.1 | 0.1 |
| BHT | Dibutylhydroxytoluene | 0.05 | 0.05 |
| Sepicide LD | Phenoxyethanol | 0.4 | 0.4 |
| Perfume | Perfume blend | 0.1 | 0.1 |
| Water | Distilled water | to 100 | to 100 |

* The dynamic viscosity of the trivent OCG: dextrin palmitate mix is 200,000 mPas (0.5 s⁻¹, 25° C.) ar 300 mPas (500 s⁻¹, 25° C.)

| | | wt % active ingredient | |
|---|---|---|---|
| Trade Name | Chemical name | Example 5 | Example 6 |
| Trivent OCG | Tricaprylin | 21.6 | 35.7 |
| Bentone 27V | Stearalkonium hectorite | 2.6 | 3.2 |
| Propylene carbonate | 1,3-dioxolan-2-one, | 0.8 | 1.0 |
| | 4-methyl- | | |
| Tween 60 | Polyoxyethylene sorbitan | 7.6 | 7.6 |
| | monostearate (20 EO) | | |
| Span 60 | Sorbitan monostearate | 1.9 | 1.9 |
| Carbopol 940 | Carbomer | | 0.3 |
| Natrosol 250 HHXR | Hydroxyethylcellulose | 1.5 | |
| Luviskol VA64W | PVP/PVA copolymer | 2.0 | 3.0 |
| Nipagin M | Methyl paraben | 0.2 | 0.2 |
| Nipasol M | Propyl paraben | 0.1 | 0.1 |
| BHT | Dibutylhydroxytoluene | 0.05 | 0.05 |
| Sepicide LD | Phenoxyethanol | 0.4 | 0.4 |
| Perfume | Perfume blend | 0.1 | 0.1 |
| Water | Distilled water | to 100 | to 100 |

1. A leave on hair care composition comprising an oil in water emulsion wherein the oil phase comprises:
i) one or more non-volatile oils, where the dynamic viscosity of any single non-volatile oil or the dynamic viscosity of a blend of non-volatile oils is less than 1000 mpa.s at 25° C. and 5 s⁻¹;
ii) an oil dispersible structurant such that the dynamic viscosity of the oil phase at 25° C. prior to formation of the emulsion is at least 50,000 mPa.s at a shear rate of 0.5 s⁻¹ and is less than 6,000 mPa.s at a shear rate of 500 s⁻¹.

2. A hair care composition according to claim 1 in which at 25° C. the oil phase, prior to formation of the emulsion has a dynamic viscosity of at least 500,000 mPa.s at a shear rate of 0.5 s⁻¹ and a dynamic viscosity of 1,000 mPa.s or less at a shear rate of 500 s⁻¹.

3. A hair care composition according to claim 1 or claim 2 in which the oil phase at 25° C. prior to formation of the emulsion has a yield stress of at least 500 Pa.

4. A hair care composition according to any preceding claim in which the oil phase prior to formation of the emulsion when sheared to a point past its yield point has a recovery ratio of at least 5%.

5. A hair care composition according to any preceding claim wherein the residue of the composition after application on hair, when part of the composition evaporates or absorbs into the hair, has rheological properties at 25° C. which are within a factor of 2 of the rheological properties of the oil phase prior to formation of the emulsion.

6. A hair care composition according to any preceding claim in which the oil phase is a liquid.

7. A hair care composition according to any preceding claim in which the non-volatile oil is selected from the group consisting of triheptanoin, tricaprylin, tricaprin, triundecanoin, trilinolein, triolein, almond oil, coconut oil, olive oil, palm kernel oil, peanut oil, sunflower oil, isopropylmyristate, isopropylpalmitate, isocetyl stearate, ethyl oleate, octyl isostearate, butyl myristate, butyl stearate, octyl palmitate, ethylhexyl cocoate, octyl dodecanol, oleyl alcohol, isostearyl alcohol, isostearic acid, mineral oil, paraffin oil, dicaprylate/dicaprate propylene glycol, C12-15 alkyl benzoate or mixtures thereof.

8. A hair care composition according to any preceding claim in which the oil structurant is selected from the group consisting of dextrin palmitate, trihydroxystearin, hydroxy stearic acid, hydrophilic hydrophobic silica, hydrophobically modified clay or mixtures thereof.

9. A hair care composition according to claim 8 in which when the structurant is a hydrophobically modified clay the oil phase further comprises a polar activator.

10. A hair care composition according to any preceding claim in which the emulsion further comprises a nonionic surfactant.

11. A hair care composition according to any preceding claim in which the level i) dispersible structurant ii) within the oil phase is from 0.5 to 20 wt % of the total oil content in the formulation.

12. A hair care composition according to any preceding claim which comprises an aqueous phase structured with an aqueous phase thickener.

13. A hair care composition according to any preceding claim further comprising a styling polymer.

14. A method of treating hair with a hair care composition comprising an oil-in-water emulsion containing a structured oil phase such that after application part of the composition evaporates or absorbs into the hair to leave a residue on the hair, where the residue has a dynamic viscosity at 25° C. of at least 50,000 mPa.s at a shear rate of 0.5 s⁻¹, and less than 6,000 mpa.s at a shear rate of 500 s⁻¹.

15. A method of treating hair according to claim 13 in which the residue at 25° C. has a yield stress of at least 500 Pa.

16. A method of treating hair according to claim 14 or 15 in which the residue when sheared to a point past its yield point has a recovery ratio of at least 5%.

17. A method of treating hair comprising the step of applying to the hair a composition as defined in any of the claims 1 to 13.

18. Use of a hair composition according to any one of claims 1 to 13 for styling hair, maintaining a style, enabling re-styling of hair, conditioning hair, reducing hair damage, providing hair shine, providing a wet look and/or controlling hair volume or alignment.
